(19) United States
(12) Patent Application Publication
(10) Pub. No.: US 2007/0212745 A1
(43) Pub. Date: Sep. 13, 2007

(54) BETA-GLUCOSIDASE AND A PROCESS FOR EXTRACTION THEREOF

(76) Inventors: Neelam Singh Sangwan, Luknow (IN); Sidharth Kumar Misra, Luknow (IN); Suman Preet Singh Khanuja, Luknow (IN); Advadesh Kumar Srivastava, Luknow (IN); Rajender Singh Sangwan, Luknow (IN)

Correspondence Address: MORGAN & FINNEGAN, L.L.P., 3 World Financial Center, New York, NY, 10281-2101, US

(21) Appl. No.: 11/531,032

(22) Filed: Sep. 12, 2006

(30) Foreign Application Priority Data
Mar. 10, 2006 (IN) 0647/DEL/2006

(51) Int. Cl. C12Q 1/34; C12N 9/24

(52) U.S. Cl. 435/18; 435/200

(57) ABSTRACT

The present invention provides a novel beta glucosidase and a process for extraction of a beta-glucosidase from Rauvolfia serpentine useful for the cleaving of beta-1,4 linkage of PNPG and to convert other gluco-conjugates such as strictosidine and raucaffricine into their corresponding aglycon such as vomilenine, commercially through immobilizing the enzyme. The β glucosidase enzyme has shown maximum activity in the acid pH range, with high optimum temperature using PNPG as substrate. The crude enzyme, when stored at 4° C., was quite stable for 6 days with 50% loss of activity. The enzyme was activated in presence of FeSO₄ in the assay mixture.

### FIELD OF THE INVENTION

The present invention relates to a novel beta-glucosidase useful for the cleavage of β 1,4 linkage of p-nitrophenyl β-D-glucopyranoside (PNPG).

More particularly, it relates to a novel beta-glucosidase which is quite stable in crude enzymatic preparation from Rauvolfia serpentina flowers, when stored at 4 degree C. for 7 days with about 54% loss of activity.

Further, the present invention relates to a process for extraction of a beta-glucosidase from Rauvolfia serpentina.

### BACKGROUND OF THE INVENTION

β-Glucosidase is a key enzyme, which imparts in hydrolysis of complex glucosides to their aglycone moieties. The enzyme works on the hydrolytic mechanism to cleave the β-1,4 linkage of β-glucosides including aryl- & alkyl- as well as diglucosides and oligosaccharides. The enzyme encompasses the wide role in biochemical and biotechnological processes and performs specifically hydrolysis of glycolipids, cellulosic biomass decomposition and cyanogenesis. The enzyme has diverse role in physiological and developmental processes in pigmentation and floral development as well as in ABA metabolism. β-Glucosidase imparts in secondary metabolites modification and their biogenesis. Production of fuel ethanol from cellulosic agricultural biomass, development of flavour and fragrance by releasing volatile components from their glucosidic precursors in fruits and vegetables.

β-Glucosidase glucohydrolase (E.C. 3;2;1;21) commonly known as β-glucosidase catalyzes the hydrolysis of a wide range of β-glucosides including alkyl- & aryl-β-glucosides, as well as diglucosides and oligosaccharides. The enzyme is localized in the lysosome of the cell, cleaves a glucose moiety from a substrate at β-1,4 linkage. The enzyme is widely used in biochemical and biotechnological processes; degradation of cellulosic biomass, hydrolysis of glycolipids, defense against microbes by cyanogenesis [Esen, A., β-D-glucosidase Biochemistry and molecular biology, Am. Chem. Soc., Washington D.C., 1993.; Bell, E. A., 1981. The physiological role(s) of secondary (natural) products. In: Stumpf P. K., Conn, E. E. (Eds.), The Biochemistry of Plants, Secondary Plant Products, Vol. 7, Academic Press, New York, pp. 1-19.; Poulton, J. E., 1990. Cyanogenesis in plants. Plant Physiol. 94, 401-405.; Phillips, D. A., Streit, W., 1996. Legume signal to rhizobial symbionts: a new approach for defining rhizosphere colonization. In: Stacey, G., Keen, N. (Eds.), Plant-Microbe Interactions, Vol. 1 pp. 236-271.]. β-Glucosidase is also associated with important developmental functions such as floral development & pigmentation [Harbome, J. B., Mabry, T. J., 1982. The flavanoids: Advances in Research, Chapman and Hall, London.; Koes, R. E., Quattrocchino, F., Mol. J. N. M., 1994. The flavanoid biosynthetic pathway in plants: function and evolution, BioEssays 16, 123-132.] and ABA metabolism [Matsuzaki, T., Koiwai, A., 1986. Germination inhibition in stigma extracts of tobacco and identification of MeABA, ABA, and ABA-β-D-glucopyranoside. Agric. Biol. Chem., 50; 2193-2199.]. β-Glucosidase also encompasses various roles in modification of secondary metabolites [Esen, A., β-D-glucosidase Biochemistry and molecular biology, Am. Chem. Soc., Washington D.C., 1993.], in production of fuel ethanol from cellulosic agricultural residues [Bothast, R. J., and B. C. Saha, 1997. Ethanol production from agricultural biomass substrates. Adv. Appl. Microbiol. 40: 261-286.; Pemberton. M. S., R. D. Brown Jr., and G. H. Emert, 1980. The role of β-glucosidase in the biocanservation of cellulose to ethanol. Can. J. Chem. Eng. 58: 723-729.; Xin, Z., Q. Yinbo, and G. Peiji. 1993. Acceleration of ethanol production from paper mill waste fiber by supplementation with β-glucosidase. Enzyme Microb. Technol. 15: 62-65.], in release of a wide variety of volatile compounds from their glucosidic precursors in fruit and vegetables [Estibalitz, O., Richard, O. A., and Ioannis, Z., 2001. The role of β-glucosidase in biosynthesis of 2,5-dimethyl-4-hydroxy-3 (2H)-furanone in strawberry. Flavour and Fragrance J., 16: 81-84.; Guegen, Y., P. Chemardin, G. Janbon, A. Amaud, and P. Galzy, 1996. A very efficient β-glucosidase catalyst for the hydrolysis of flavour precursor of wines and fruit juices. J. Agric. Food. Chem. 44: 2336-2340.; Shoseyov, O., B. A. Bravdo, R. Ikon, and I. Che, 1990. Immobolized endo-β-glucosidase enriches flavour of wine and passion fruit juice. J. Agric. Food Chem., 27: 1973-1976.], in conversion of storage form of cytokinin to its active form [Smith, A. R., Van Staden, J., 1978. Changes in endogenous cytokinin levels in kernel of Zea mays L. during imbibition and germination. J. Exp. Bot., 29: 1067-73.], in development of some flavour compounds such as monoterpenols, C-13 norisoprenoids and shikimate derived compounds accumulates in fruits as non flavour precursor linked to mono- or di-glycosides before enzymatic hydrolysis [Vasserot, Y., A. Amaud, and P. Galzv, 1995. Monoterpenyl glycosides in plant and their biotechnological transformation. Acta. Biotechnol. 15: 77-95.; Winterhalter, P., and G. K. Skouroumounis, 1997. Glycoconjugated aroma compounds: occurrence, role and biotechnological transformation. Adv. Biochem. Eng. Biotechnol. 55: 73-105.].

Rauvolfia serpentina is a fairly wide spread plant in tropical part of Himalayas, the Indian peninsula, Sri Lanka, Burma and Indonesia. The plant is also cultivated in Thailand, Malay Peninsula, Sumatra, Java and the Lesser Sunda Island. The application of β-glucosidase in Rauvolfia serpentina is well understood in metabolic pathway of indole alkaloids. Monoterpenoid indole alkaloids are a vast and structurally complex group of plant compounds and rarely occur as glycosides. In the biosynthesis and metabolism of alkaloids of the ajmalan-group, which are characteristic of the traditional medicinal plant Rauvolfia, two glucosides are involved as intermediates. The biosynthetic intermediate strictosidine, formed from tryptamine and secologanin by the enzyme strictosidine synthase. Strictosidine occupies a central position in the synthesis of all monoterpenoid indole alkaloids in plant genera such as Catharanthus, Rauvolfia, Strychnos, Cinchona, etc. In this case, the glucose moiety functions as a protecting group to stabilize the molecules. After deglucosylation of strictosidine by strictosidine glucosidase, the highly reactive and unstable aglycone is directed into different biosynthetic pathways that species dependant and result in the production of various indole and quinoline alkaloids. Accumulation of raucaffricine, a glucoside of vomilenin which is a direct biosynthetic precursor of ajmaline. Vomilenine glucosyltransferase (VGT) forms raucaffricine from vomilenin, resulting in the accumulation of this glucoside and represents a part of one of the most abundant indole alkaloids. This side product of ajmaline biosynthetic pathways is deglucosylated by another highly specific enzyme from Rauvolfia—raucaffricine glucosidase (RG, EC 3.2.1.125). Therefore, raucaffricine biosynthesis and the re-utilization of it for the ajmaline biosynthetic pathway could be the crucial and rate limiting step for the formation of ajmaline. For that reason, both enzymes, VGT and RG, are of an interest.

Present invention provides a novel beta-glucosidase of plant origin possessing unique combination of optimum temperature of 60 degree C., activation by FeSO₄ and stability with about 46% activity for 7 days. It is totally different from cultured cells R. serpentina cells into a bacterium E. coli (Irina gerasimenko, uri Sheludko, Xueyan Ma and Joachim Stockigt, 2002, Eur. J. Biochem. 269, 2204-2213) in terms of its substrate, optimum temperature of activity and its natural plant flower as source. The reported enzyme is cloned and expressed in E. coli. The enzymatic/metabolic complement of a cultured plant cells is far different from native tissue expression. Therefore, enzyme of the present invention does not share its properties to the cultured cells and is novel not reported so far including from R. serpentina.

There is no prior art known so far, for isolation of β-glucosidase using present process. Therefore this way of extraction of enzyme seems to be advantageous over others in the frame of less time and high activity content.

### OBJECTS OF THE INVENTION

The main object of the present is to provide a novel beta-glucosidase useful for the cleavage of β 1,4 linkage of p-nitrophenyl β-D-glucopyranoside (PNPG).

Another object of the present invention is to provide a novel beta-glucosidase, which is quite stable in crude enzymatic preparation from Rauvolfia serpentina flowers, when stored at 4 degree C. for 7 days with about 54% loss of activity.

Further, another object of the present invention is to provide a novel beta-glucosidase useful to convert other gluco-conjugates such as strictosidine and raucaffricine into their corresponding aglycon such as vomilenine, commercially through immobilizing the enzyme.

Yet another object of the present invention is to provide a novel beta-glucosidase having maximal catalytic activity at 60° C.

Still another object of the present invention is to provide a process for extraction of a beta-glucosidase from plant tissues of Rauvolfia serpentina.

### SUMMARY OF THE INVENTION

The present invention deals with beta-glucosidase as well as a process for extraction thereof from Rauvolfia serpentina useful for the cleaving of beta-1,4 linkage of p-nitrophenyl β-D-glucopyranoside (PNPG) and to convert other gluco-conjugates such as strictosidine and raucaffricine into their corresponding aglycon such as vomilenine, commercially through immobilizing the enzyme. The said enzyme is highly active at temperature 60 degree C. in the presence of FeSO₄.

### DETAILED DESCRIPTION OF THE ENZYME

Accordingly, the present invention provides a novel β glucosidase enzyme useful for the cleavage of β 1,4 linkage of p-nitrophenyl β-D-glucopyranoside (PNPG).

In an embodiment of the present invention, the said enzyme has following characteristics:
a) it is stable for more than 7 days at 4 degree C.;
b) it is active in acidic pH range;
c) optimum activity of this enzyme is at about 60 degree C.;
d) it is strong active in the presence of FeSO₄;

In another embodiment of the present invention, the said enzyme is obtained from the extract of plant tissues of Rauvolfia serpentina.

Further, in an embodiment of the present invention, the plant tissue used is a flower of Rauvolfia serpentina.

Further, the present invention also provides a process for extraction of a beta-glucosidase from plant tissues of Rauvolfia serpentina wherein the said process comprising the steps of:
a) homogenizing the plant tissue in a cold extraction medium in the ratio ranging from 1:1 to 1:3 (w/v)
b) centrifuging the homogenized solution obtained from step (a) at 10,000 rpm to 12,000 rpm for 20-30 min 4 degree C. to 10 degree C. to obtain the clear supernatant containing desired product.

In an embodiment of the present invention, the used plant tissue from Rauvolfia serpentina is selected from the group consisting of roots, stem, old, mature and young leaves, flower stem (petiole), flowers and fruits etc.

In another embodiment of the present invention, the flowers have maximum specific activity of the enzyme.

Further, in another embodiment of the present invention, the extraction medium used is comprises disulphide bond reducing agent and chelating agent in phosphate buffer at pH about 6.0 in the ratio ranging from 1:5 to 2:5.

In yet another embodiment of the present invention, the reducing agent used is beta mercaptoethanol.

In yet another embodiment of the present invention, the chelating agent used is EDTA.

The extraction of the glucosidase enzyme was optimized with respect to buffer, pH, protecting and stabilizing agents to obtain maximum extractable activity. All the operations of enzyme isolation were carried out at 0-4° C. unless specified otherwise.

The extraction medium of present invention also relates to a process for the extraction of a novel β-glucosidase from plant tissues from a natural source and useful for the cleaving of β-1,4 linkage of PNPG (p-nitrophenyl β-D-glucopyranoside), said process comprising: homogenizing the plant tissue in a cold extraction medium (1 g tissue/3 ml). Flowers of Rauvolfia serpentina were homogenized in 100 mM Potessium-phosphate buffer (pH 6.0) including 10 mM β-Mercapto ethanol and 2 mM E.D.T.A. (Ethylene Diamine Tetra Acetic Acid). The tissue homogenate was centrifuged at 12,000×g for 30 min at 4° C. and the clear supernatant was collected which was used as the enzyme source.

The following examples are given by way of illustration of the present invention and should not be construed to limit the scope of present invention.

### EXAMPLE 1

### Preparation of Crude Extract:

Rauvolfia serpentina (Sarpgandha) plants were raised from root cuttings at the glass house of Central Institute of Medicinal and Aromatic Plants (CIMAP), Lucknow by following standard agronomic practices. The extraction of the glucosidase enzyme was optimized with respect to buffer, pH, protecting and stabilizing agents to obtain maximum extractable activity. All the operations of enzyme isolation were carried out at 0-4° C. unless specified otherwise. The extraction medium of present invention also relates to a process for the extraction of a B glucosidase from plant tissues from a natural source and useful for the cleaving of β-1,4 linkage of PNPG, said process comprising: homogenizing the plant tissue in a cold extraction medium (1 g tissue/3 ml). Flowers of Rauvolfia serpentina were homogenized in 100 mM K-phosphate buffer (pH 6.0) including 10 mM β-Mercapto ethanol and 2 mM E.D.T.A. (Ethylene Diamine Tetra Acetic Acid). The tissue homogenate was centrifuged at 12,000×g for 30 min at 4° C. and the clear supernatant was collected. Assays specific for the determination of optimum pH, optimum temperature, stability and activation were performed. The enzyme was found to be most active in the acid pH range of 5, and showed maximal activity temperature 60° C.

### EXAMPLE 2

### β-Glucosidase Assay:

Enzyme activity was assayed using the modified method described by Stevens et al [19], assay mixture in a total volume of 200 μL contained 2.5 mM p-nitrophenyl β-D-glucopyranoside in 100 mM Citrate-Phosphate buffer (pH 5) at defined temperatures with 10 μL of enzyme preparation. After 15 min of incubation time the reaction was stopped by adding 800 μL of 1M Sodium carbonate and the absorbance of p-nitrophenol (the product of reaction) was measured at wavelength 405 nm.

Calculation of Units: The enzyme activity was expressed in terms of μmole of p-nitrophenol formed per minute using the molar extinction coefficient (E) 18,350 m⁻¹ cm⁻¹ [Estibalitz, O., Richard, O. A., and Ioannis, Z., 2001. The role of β-glucosidase in biosynthesis of 2,5-dimethyl-4-hydroxy-3 (2H)-furanone in strawberry. Flavour and Fragrance J., 16: 81-84.]

TABLE 1
Stability assessment of novel β-glucosidase from Rauwolfia serpentina
flower

| S. No. | Duration | % Activity |
|---|---|---|
| 1 | 2 Hr. | 100 |
| 3 | 6 Hr. | 97.7 |
| 4 | 24 Hr (one day) | 82.2 |
| 5 | 48 Hr (two days) | 73.3 |
| 6 | 72 Hr (three days) | 65.5 |
| 7 | 96 Hr (four days) | 55.0 |
| 8 | 144 Hr (six days) | 50.0 |
| 9 | 168 Hr (seven days) | 46.7 |

TABLE 2
Effect of FeSO4 on β glucosidase

β glucosidase

| S. | Conc. | | Fold activity |
|---|---|---|---|
| No. | (mM) | Activity (I.U.) | enhanced |
| 1 | 0 | 41.41 | 1 |
| 2 | 1 | 71.93 | 1.74 |
| 3 | 5 | 335.69 | 8.11 |
| 4 | 10 | 627.79 | 15.16 |

Protein estimation was done by the method of Lowry (1951) using bovine serum albumin as reference standard [Lowry, O. H. Rosenbrough, N. J., Farr, A. L. and Randall, R. J. (1951). J. Biol. Chem. 193: 265.]

TABLE 3
β-Glucosidase activity versus protein concentration

| Enzyme Volume | Protein/Assay | |
|---|---|---|
| (μL) | (μg) | I.U./Assay |
| 2 | 11.532 | 0.392 |
| 5 | 28.880 | 0.708 |
| 10 | 57.760 | 1.144 |
| 20 | 115.520 | 1.634 |
| 30 | 173.280 | 3.269 |
| 40 | 231.040 | 3.160 |
| 50 | 288.800 | 2.179 |
| 75 | 433.200 | 2.179 |
| 100 | 577.600 | 1.089 |

### ADVANTAGES

The main advantages of the present invention are:

1. The glucosidase enzyme from crude preparation of Ravolfia serpentina flowers has shown the capability to cleave β-linkage of PNPG.

2. The enzyme from flowers showed most of the activity in the acidic pH range and the activity is substantially lost in the neutral and alkaline pH range, with optimum activity at pH 5 (100 m M Citrate-phosphate buffer) and temperature 60° C.

3. The enzyme activity increased linearly with protein concentration up to 170 ug per assay.

4. Enzyme was found to be quite stable in crude enzymic preparation from Rauvolfia serpentina flowers, when stored at 4° C. for 7 days, with about 54% loss of activity.

5. The enzyme from Rauvolfia serpentina flowers is unique in its high optimum temperature 60° C. and its activation by FeSO4.

We claim:

1. A β glucosidase enzyme useful for the cleavage of β 1,4 linkage of p-nitrophenyl β-D-glucopyranoside (PNPG).

2. A β glucosidase enzyme as claimed in claim 1, wherein the said enzyme have following characteristics:
a) it is stable for more than 7 days at 4 degree C.;
b) it is active in acidic pH range;
c) optimum activity of this enzyme is at about 60 degree C.;
d) it is strong active in the presence of FeSO₄;

3. A β glucosidase enzyme as claimed in claim 1, wherein the said enzyme is obtained from the extract of plant tissues of Rauvolfia serpentina.

4. A β glucosidase enzyme as claimed in claim 1, wherein the plant tissue used is a flower of Rauvolfia serpentina.

5. A process for extraction of a β-glucosidase from plant tissues of Rauvolfia serpentina wherein the said process comprises:
a) homogenizing the plant tissue in a cold extraction medium in the ratio ranging from 1:1 to 1:3 (w/v)
b) centrifuging the homogenized solution obtained from step (a) at 10,000 rpm to 12,000 rpm for 20-30 min at a temperature in the range 4 degree C. to 10 degree C. to obtain the clear supernatant containing desired product.

6. A process as claimed in claim 5, wherein the used plant tissue from Rauvolfia serpentina is selected from the group consisting of roots, stem, old, mature and young leaves, flower stem (petiole), flowers and fruits etc.

7. A process as claimed in claim 5, wherein the extraction medium used is comprises a disulphide bond reducing agent and a chelating agent in phosphate buffer at pH of about 6.0 in the ratio ranging from 1:5 to 2:5.

8. A process as claimed in claim 5, wherein the reducing agent used is beta mercaptoethanol.

9. A process as claimed in claim 5, wherein the chelating agent used is EDTA.
